(19) United States
(12) Patent Application Publication
(10) Pub. No.: US 2004/0214313 A1
(43) Pub. Date: Oct. 28, 2004

(54) Cell interaction culture system and uses thereof

(76) Inventors: Weihua Zhang, Houston, TX (US); Xin Ma, Houston, TX (US)

Correspondence Address: Hon-Man Lee, Law Office of Hsyung & Associates, Suite 109, 7001 Corporate Drive, Houston, TX, 77036, US

(21) Appl. No.: 10/622,762

(22) Filed: Jul. 20, 2003

Related U.S. Application Data
(60) Provisional application No. 60/466,218, filed on Apr. 28, 2003.

(51) Int. Cl. C12M 1/34; C12M 1/22

(52) U.S. Cl. 435/288.4; 435/305.2; 435/29

(57) ABSTRACT

The present invention relates to a novel cell co-culture system and methods of using the same for studying cellular interactions. The co-culture system comprises a cell culture container partitioned into multiple compartments by removable partition units/separating walls. Different cell types or materials can be placed and cultured in the various compartments. When the cells are ready for cell interaction studies, the removable walls separating two adjacent compartments are removed and cellular interaction can then be studied in a defined area, i.e. the area previously occupied by the bottom edges of the removable walls. The present invention thus provides a cell co-culture system that would set up a new and flexible platform for most cell interaction studies.

### BACKGROUND OF THE INVENTION

1. Field of the Invention

The present invention relates generally to apparatus and methods of in vitro cell culture. More specifically, the present invention provides a cell co-culture system that allows culturing different types of cells on the same culture container and studying cell-cell interactions in defined areas. This cell culture system invented herein is named ‘MaxZon Cell-X Co-culture System’.

2. Description of the Related Art

With increasing interests in studying cell-cell interaction, there is a growing need for a convenient cell co-culture system that would allow culturing different types of cells on the same culture container and studying cell-cell interaction at pre-determined time and space. It is desirable to have a cell co-culture system that would allow treating the different interacting cells with different reagents separately prior to cell-cell interaction. Preferably, the co-culture system should also allow easy temporal monitoring of cellular and molecular transformations that take place in the interacting cells. However, currently there is no satisfactory cell co-culture system that would meet the above-mentioned criteria. Commercialized cell co-culture systems for cell interaction studies all have major pitfalls, e.g. it is impossible to use them to study interactions among a variety of morphologically indistinguishable cells or to treat the interacting cells in individualized conditions prior to cell-cell interaction. The present invention fulfills this need and desire in the art and provides a novel cell co-culture system that would set up a new and flexible platform for most cell-cell interaction studies. This will greatly improve data production in areas of cutting edge biomedical research such as cancer metastasis, developmental biology, toxicology, in vitro diagnosis, and infectious disease.

### SUMMARY OF THE INVENTION

It is an object of the present invention to provide a novel cell co-culture system for studying cellular interactions such as cell-cell, cell-tissue or cell-solid biological materials interaction. The present invention allows culturing different types of cells on the same container and studying cellular interactions in defined areas. This system comprises an ordinary cell culture container such as culture dish, multi-well culture plate or culture chamber slide (FIG. 1) partitioned into several compartments by multiple removable partition units. In its simplest form, the partition unit is an open-ended straight wall (FIG. 2A). One or both of its open ends can be attached to the side wall of a cell culture container (see FIGS. 1C, 3). In another embodiment, the partition unit can be cylindrical or rectangular in shape, thereby enclosing a circular or rectangular area on the tissue culture plate (FIG. 2B, 2D).

The partition units are delicately adhered to the culture surface of the cell culture container by cell-safe material such as medical grade silicone glue, or attached and sealed to the inner surface by pressure seal. Alternatively, the partition units can be attached to the cell culture surface by direct and tight contact between optically polished surfaces of the partition units and the cell culture container (without sealing materials).

In one embodiment of the present invention, a cell culture dish is separated into multiple compartments by removable partition units (FIG. 3). The partition units converge to a centrally located culturing cylinder. This central cylinder defines a central culture area that allows one type of cell to grow and interact with cells in the surrounding compartments, or allows various cell populations growing in the surrounding compartments to interact (multiple interactions).

Different types of cell or materials can be placed and cultured in various compartments of the cell co-culture system described herein. When the cells are ready for cell interaction studies, the removable partition(s) separating two adjacent compartments is (are) removed. Removing the partition unit(s) and the adhesive silicone (or pressure seal) leave no damage on the surface of the culture container and cells in the adjacent compartments can interact with each other in a defined area, i.e. the area previously occupied by the bottom edge of the removable partition unit(s).

The biological events that take place between the interacting biological materials can be studied in situ. More significantly, the present invention makes it possible to conduct cell-cell interaction studies involving multiple morphologically indistinguishable cell types on a solid support, which is impossible using currently available cell co-culture methods. In addition, the present invention provides a convenient disposable cell sampler that can remove cells from specific areas on the culture container for further downstream studies.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1. Preferable common cell culture containers used in MaxZon Cell-X Co-culture system: A, a cell culture dish; B, a cell culture plate; C, a cell culture chamber slide. Gray areas represent the bottom of a cell culture container.

FIG. 2. Preferable removable partition units of MaxZon Cell-X Co-culture system. A, open straight wall with sealing material on the bottom edge of the wall; B, a closed cylinder wall with/without sealing material on the bottom edge of the wall; C, combinations of a open straight wall and a cylinder wall; D, a partition units enclosing a rectangular area. The enclosed rectangular area can be further divided into multiple compartments by partitions as shown in A. The bottoms of B and D can be optical polished so that they can form tight contacting seal with the culture surface of a cell culture container without using sealing material. In cases where no sealing material is used, holders attached to the partition units are used to secure the position of the removable partitions on the cell culture container.

FIG. 3. Top view of MaxZon Cell-X Co-culture System. 1. Culture container body (a culture dish serves as an example). The gray area represents the bottom of the container and each compartment can be marked with letters (container cover is omitted from this figure). 2. Removable partition unit. The cell culture container can be divided into any numbers of compartments by the partition units according to the user's choice. 3. Removable central culturing cylinder. 4. Sealing part of the partition unit. The width and shape of the areas covered by the bottom edge of the partition units are optimized for individualized cell interactions. 5. Overhang of the sealing part. These parts serve as holders for tearing off the sealing part inside the culture container.

FIG. 4. Bottom view of MaxZon Cell-X Co-culture System. 1. Culture container body. The gray area represents the bottom of the container (container cover is omitted from this figure). 2. Removable partition unit. 3. Removable central culturing cylinder. 4. Sealing part of the central cylinder. 5. Overhang of the sealing part. 6. Marking lines of the interacting areas. 7. Central culture area of the culture container.

FIG. 5. Top view of MaxZon Cell-X Co-culture System after one partition unit is removed. A and B are two adjacent compartments.

FIG. 6. Top view of the MaxZon Cell-X Co-culture System after tearing off the sealing part of the partition unit.

FIG. 7. Top view of the MaxZon Cell-X Co-culture System, showing interaction area between compartments A and B. After removal of both the partition unit and its sealing material, the two compartments A and B are re-connected. The blank area, the interaction area, is clean and ready for cells (or tissues) in compartments A and B to interact.

FIG. 8. Top view of MaxZon Cell-X Co-culture System after removing all partition units and the central culturing cylinder. After removing all the partitions and the central culturing cylinder, the bi-interaction area is the place where cells in adjacent compartments will interact. Multiple interactions among various cells will take place in the blank central area that was defined by the walls of the central culturing cylinder.

FIG. 9. Top view of the bi-interaction area between compartments A and B for mutual growth impact studies. The edge of compartment A is sawtooth and the edge of compartment B is straight (FIG. 9A). After removal of both the partition and sealing material, the two compartments A and B are re-connected. The blank area is clean and ready for cells (or tissues) in compartments A and B to migrate toward each other. If cells growing or material placed in compartment A has no impact on the growth of cells in compartment B, their growth front lines will show pattern depicted in FIG. 9B. If cells growing or material placed in compartment A has inhibitory impact on the growth of cells in compartment B, their growth front lines will show pattern depicted in FIG. 9C. If cells growing or material placed in compartment A has inducible impact on the growth of cells in compartment B, their growth front lines will show pattern depicted in FIG. 9D.

FIG. 10. Schematic representation of a cell sampler of MaxZon Cell-X Co-culture System. In general, the cutting end and the sampling end can be in the shape of square, rectangle or circle.

### DETAILED DESCRIPTION OF THE INVENTION

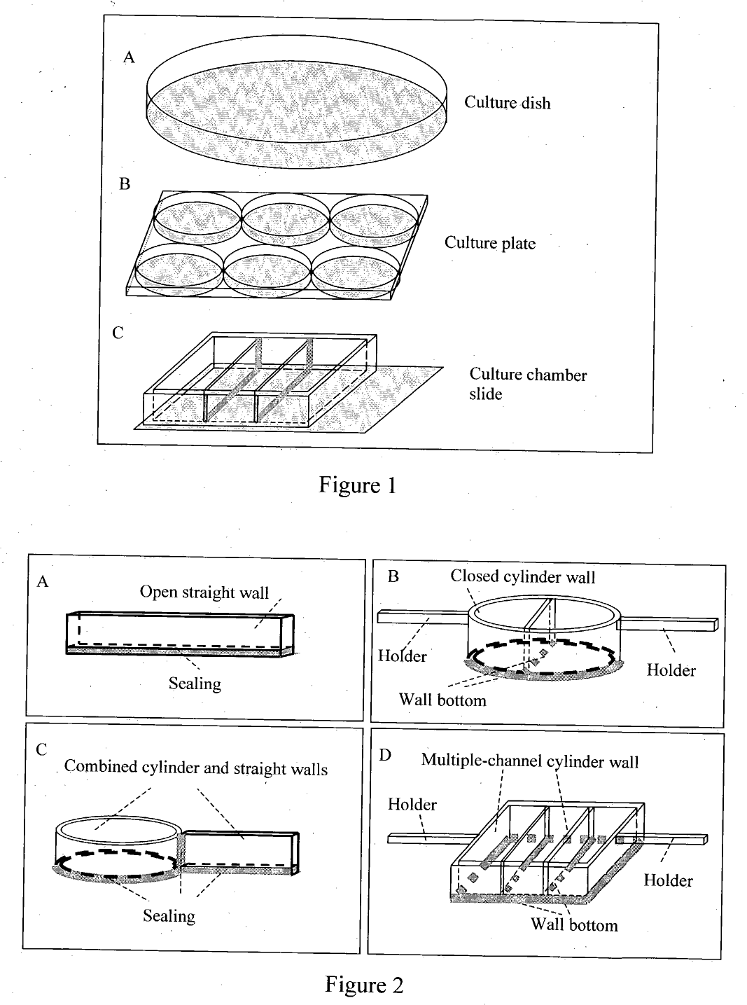

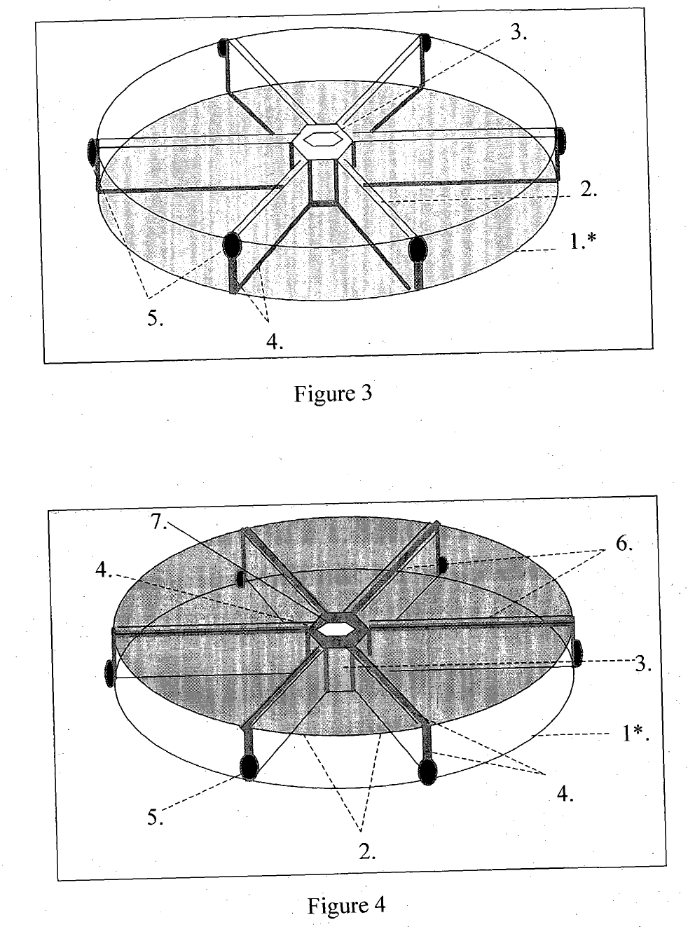

The central features of the present invention are the removable partition units and the cell-interacting areas defined by these removable partition units. FIGS. 1 and 2 depict different embodiments of cell culture containers and removable partition units of the present invention. FIGS. 3 to 9 demonstrate one embodiment of the present invention as a cell culture dish containing a removable central cylinder and removable partition units.

As used herein, “cell culture container” refers generally to commonly used cell culture apparatus such as cell culture dish, multi-well culture plate and cell culture chamber slide (FIG. 1). The shape, volume and the material of the culture container can vary to meet different cell culture requirements.

As used herein, “removable partition unit” refers to a removable partition or separating wall that divide a cell culture container into various compartments. The co-culture system of the present invention may include single or multiple partition unit(s) that partition a cell culture container into two or more compartments into which different cells can be cultured. The simplest form of a partition unit is an open-ended straight wall (FIG. 2A). It can also be a combination of a cylinder and a straight wall (FIG. 2C). Alternatively, the partition unit can be cylindrical or rectangular in shape (FIG. 2B, 2D), thereby enclosing a circular or rectangular area on the cell culture container.

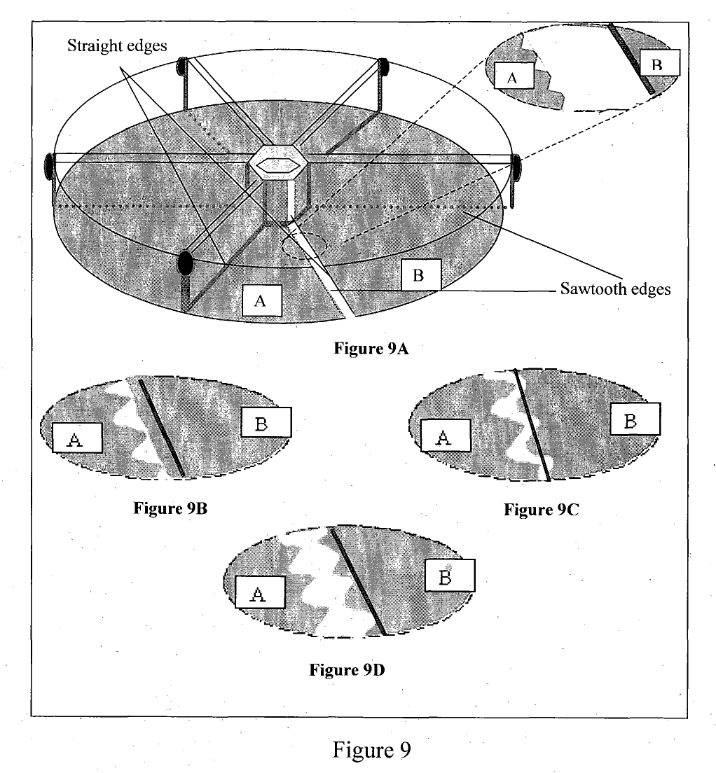

In addition to dividing a cell culture container into different compartments, the partition unit also defines an area on the cell culture container for cell-cell interaction. This area is the area covered by the bottom edge of a partition unit. The removable partition unit is attached and sealed on the cell culture surface of a cell culture container by a type of sealing glue (medical grade silicone glue, etc.), a pressure seal, or by a direct tight contacting seal between optically polished bottom surface of a partition unit and the surface of the cell culture container. Once the partition unit(s) is (are) removed, the area(s) on the culture container previously covered by the bottom edge(s) of the partition(s) will serve as area(s) for cell-cell interaction. The areas covered by the bottom edges of the partition units can be delineated by marking lines on the outside surface of the culture container to facilitate cell-cell interaction monitoring (FIG. 4). The bottom edge of a partition unit can be fabricated into various thickness and shapes (for example, cuneiform or sawtooth; see FIG. 9) so that the area covered by the partition unit, and hence the area for cellular interaction, can be varied to fulfill a variety of purposes.

As used herein, “central culturing cylinder” refers to an optional, cylindrical-shaped partition unit located in the middle of a culture dish (see FIG. 3). The central culturing cylinder serves as another cell culture compartment and is removable. If the central culturing cylinder is removed, multiple interactions between the cells growing in the central area and those cells in the surrounding compartments can take place in the areas that are defined by the wall of the central culturing cylinder.

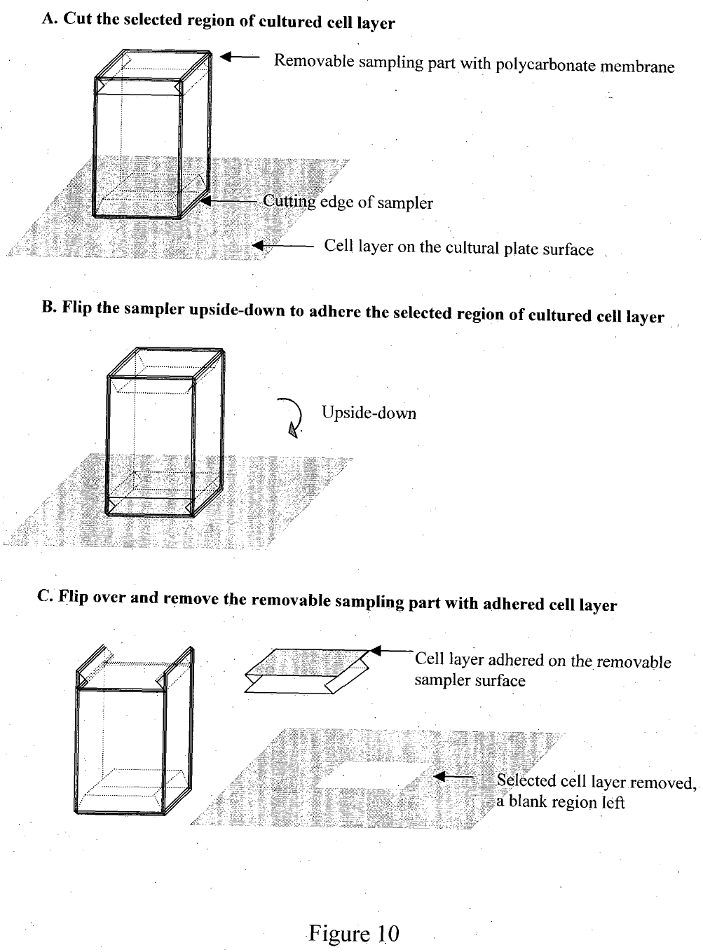

As used herein, a “cell sampler” refers to an optional and disposable sampler comprising two ends, a cutting end and a sampling end (FIG. 10). The cutting end is used to select a target region on a cultured cell layer, and clears the edges of the selected region. The cutting edge can be sharp or of certain thickness of blunt edges. Once a target region is selected on a cultured cell layer, the cells within that region can be collected by the sampling end. The sampling end comprises a removable part with a pretreated surface (e.g. a polycarbonate membrane, or poly-lysine treated microscopic slide cover slip) to which the cells in the targeted area can adhere. Further analysis or experiments can be conducted on the membrane carrying the attached cells. In general, the cutting end and the sampling end can be in the shape of square, rectangle or circle, etc.

The cell co-culture system of the present invention would significantly fulfill the needs of cellular interaction studies by presenting the following advantages:

1. It is easy to distinguish the interacting cells during cell co-culture without using any biological marker because the cell co-culture system provides clear physical locations (individual compartments) and interfaces (the gap areas) for cell culture and cellular interactions.

2. It is easy to monitor the cell interactions because cellular interactions only takes place in defined areas, i.e. the gap areas, areas that are covered by the partition units.

3. It is easy to study cell interactions among multiple cell types. In the case of using a culture dish, the central compartment (area defined by the central culturing cylinder) can serve as a common area where two or more cell types on the culture dish can interact.

4. The starting time of cellular interaction can be easily controlled by removing the partition units at a time when the cells or tissues in the different compartments of the culture container are ready to interact.

5. It is easy to constantly monitor live cell migration and interaction.

6. It is easy to conduct downstream analyses on the interacting cells. Since interaction between the cells happens in clearly defined areas, downstream analyses such as immunohistochemical or gene expression analysis can be easily targeted. In addition, the convenient sampler can take selected cells off from the cultured cell layer for time dependent analysis.

7. It is an unprecedented system to study relative migrations between different types of cells.

8. It is a friendly system for manufacturers and users of cell co-culture facilities.

Thus, the present invention is directed to a cell co-culture system comprising a cell culture container partitioned into two or more compartments by one or more removable partition units. The bottom edge of the removable partition unit(s) is in contact with the cell culture surface of the cell culture container, and different types of cells can be cultured in each of the compartments. Cellular interaction or cell migration is to be observed in an area on the cell culture container covered by the bottom edge(s) of the removable partition unit(s).

Preferably, the cell culture container can be a culture dish, a multi-well culture plate, or a culture chamber slide (FIG. 1). The diameter or the diagonal dimension of the cell culture container is from about 10 mm to about 300 mm. In general, the cell culture container is made of polystyrene, glass, or plastic with high optical compatibility of glass. The inner cell culture surface of the culture container can further be treated or covered with some membranes or biomaterials such as extracellular matrix, polycarbonate membrane or solid culture media to meet special cell culture requirements.

Preferably, the removable partition unit is made of polystyrene, glass, medical grade silicon, metal, or biomembranes with different permeabilities for specific studies. The thickness of the removable partition unit, as well as the width of the area covered by the bottom edge of a removable partition unit on a cell culture container, is from about 0.01 mm to about 10 mm. The two sides on the bottom edge of a removable partition unit can be fabricated in a shape of straight line, sawtooth-shaped or wave-shaped (see FIG. 9).

In general, the partition unit is attached to the cell culture surface of the culture container. In one embodiment, the partition unit is sealed to the culture container with sealing material that are biologically inert, removable and leave no damage on the inner surface of the cell culture container. Representative examples of such sealing material include sealing glue or pressure seal that comprises medical grade silicone glue or rubber. Medical grade silicone glue vulcanizes at room temperature, and the vulcanized silicone can be easily removed and leaves no damage to the culture container. Alternatively, the partition unit can be attached to the cell culture container without using any sealing material. In this latter case, the partition unit is attached to the cell culture container by direct and tight physical contact between optically polished surfaces of the partition unit and the surface of the culture container.

In one embodiment of the present invention, the co-culture system comprises a culture dish partitioned into multiple compartments by removable partition units or separating walls. As shown in FIGS. 3 and 4, the partition units/separating walls 2 are connected to a central removable culturing cylinder 3. The central culturing cylinder serves as another cell culture compartment. If the central culturing cylinder is removed, multiple interactions between the cells growing in the central area and those cells in the surrounding compartments can take place in the areas that are defined by the wall of the central culturing cylinder. The bottom edge of each removable partition unit/separating wall is attached to the cell culture surface of the culture dish by a sealing material 4 that is connected to an overhang 5 located on the exterior or interior side wall of the culture dish. The overhangs serve as handles for tearing the sealing material off the culture dish. Preferably, the sealing material is biologically inert and removable from the culture dish without damaging the culture dish, e.g. medical grade silicone glue. The co-culture system can further include a cell sampler as described above.

In another embodiment of the present invention, the co-culture system comprises removable partition unit that is cylindrical or rectangular in shape (FIGS. 2B, 2D), thereby enclosing a circle, a rectangle or a square on the cell culture container. In this embodiment, the partition unit is attached to the cell culture container by tight physical contacting seal (without adhering and sealing material) through contact between optically polished surfaces of the partition unit and the surface of the cell culture container. To prevent accidental moving of the partition unit during cell culture, the partition is attached to holders that bridge the gaps between the partition and the side wall of the cell culture container (FIGS. 2B, 2D). It should also be noted that the area enclosed by the partition can further be divided into smaller compartments by additional separating walls (FIG. 2D).

In another embodiment of the present invention, there is provided a method of studying cell-cell interaction or interaction between a cell type and a solid biological material. The method involves first culturing different cell types or solid biological material in the cell co-culture system disclosed herein, wherein the cells or solid biological material are placed in different compartments of the culture container. After removing the partition units and sealing material (or just the partition units when no sealing material is used) from the cell culture container; cell-cell interaction or interaction between a cell type and a solid biological material can be examined in the areas covered previously by the partition units. This method can further involve using the cell sampler provided herein to collect selected cells on the culture container. Selected region on the culture container can first be marked by the cutting end of the cell sampler, then the cells in the selected region can be collected by adhering to a removable cell sampling surface attached to the sampling end of the cell sampler.

In yet another embodiment of the present invention, the co-culture system can be used to study relative migrations between different types of cells under the same conditions. Different types of cells are cultured in different compartments of the cell culture container, and cell migration is then examined in the area covered previously by the bottom edges of the removable partition units.

The co-culture system of the present invention can also be used to study mutual, growth impacts of different types of live cells. Different types of cells are cultured in different compartments partitioned by removable partition units that have one side of their bottom edges in straight line and the other sides sawtooth-shaped. Monitoring the interacting front lines of cells in the area covered previously by the bottom edges of the removable partition units would reveal mutual growth impacts of these cells.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### EXAMPLE 1

Method of Cell Co-Culture Studies

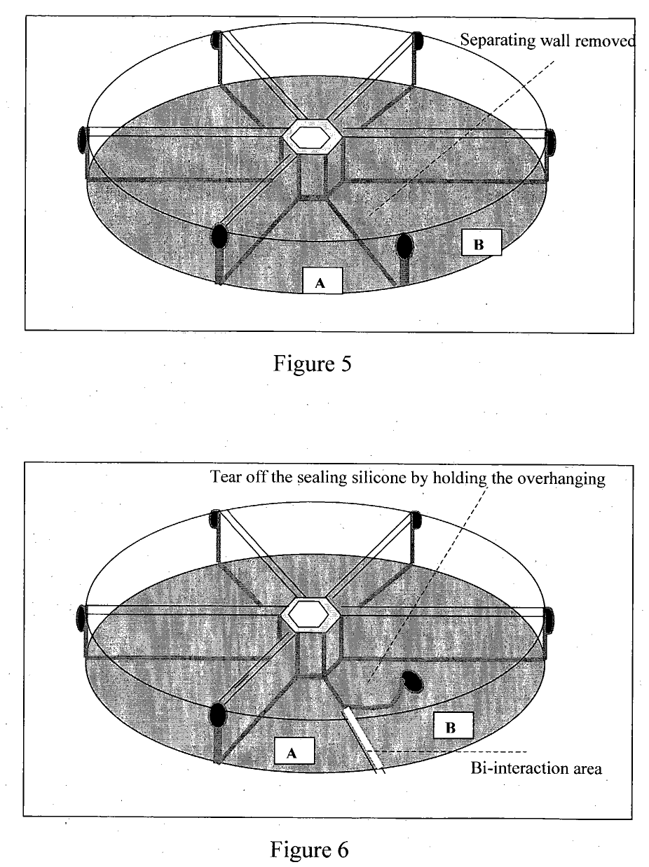

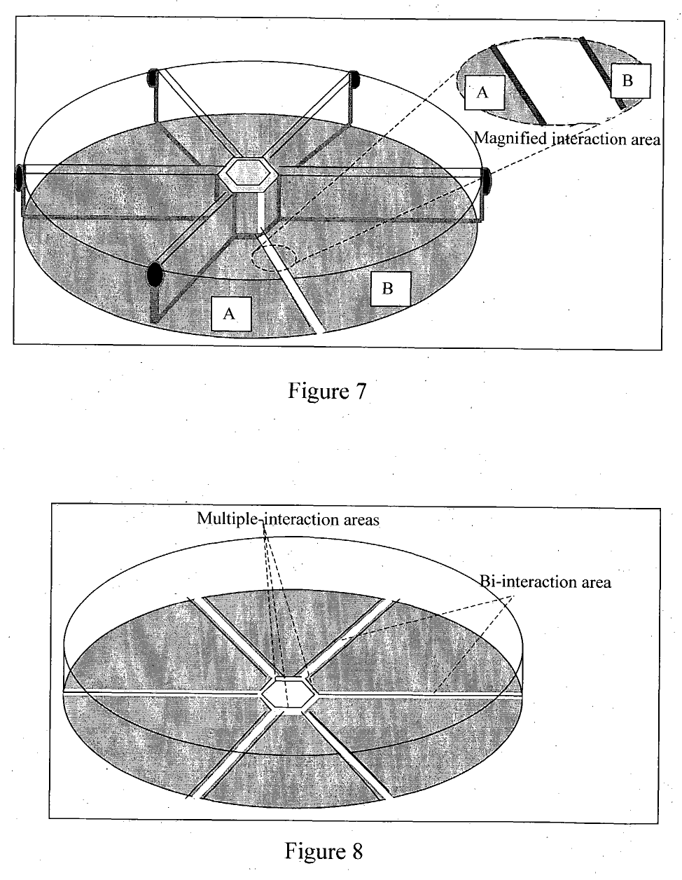

In one embodiment of the present invention, a cell culture dish is used for cell culture. Different cell types or materials can be placed and cultured in the various compartments of the cell co-culture system (FIG. 3). When the cells are ready for cell interaction studies, the removable partition unit(s) separating two adjacent compartments is removed (FIG. 5). The sealing material (e.g. medical silicone glue) that affixes the separating wall to the culture container can then be torn off using the overhang as a handle (FIG. 6). Removing the partition units and the adhesive silicone leaves no damage on the surface of the culture container and cells in the adjacent compartments can interact with each other in a defined area, i.e. the area previously occupied by the bottom edges of the removable separating walls (FIG. 7). The central culturing cylinder can also be removed. Multiple interactions between the cells growing in the central area and those cells in the surrounding compartments can take place in the areas that are occupied previously by the walls of the central culturing cylinder (FIG. 7).

Moreover, the present invention provides a cell sampler capable of marking and collecting selected cells on the culture container (FIG. 10). The cutting end of the sampler is used to select and mark a region on the cultured cell layer. After a region of cells is selected, the sampler is flipped over so that a removable biomembrane attached to the sampler end will adhere and collect all the cells within the selected region.

### EXAMPLE 2

Applications of Cell Co-Culture Studies

Interactions between tumor cells and different tissue cells are crucial points in the study of tumor malignancy. The present co-culture system is useful for studies aimed at understanding the molecular mechanisms behind tissue-specific metastasis of specific cancer cells. One or several kinds of cancer cells and cells originated from target/non-target tissues for these cancer cells can be co-cultured in different compartments of the present co-culture system. When the separating walls are removed, these cells will interact in defined area under the same culture conditions. Differences of biological events that happen in/between the cancer cells and their metastatic (or non-metastatic) target cells can then be studied.

In another embodiment, the present co-culture system is useful for studies of organ development. Organs in the body are each composed of several kinds of cells. The orientation of specific cells in an organ depends on cell-cell interaction during organ development. These cell-cell interactions induce cell differentiation, proliferation and even dedifferentiation. The present cell co-culture system provides researchers a new and convenient tool to study cellular interactions during organ development. For example, epithelial cells can be cultured in one compartment, whereas stromal cells such as fibroblasts or smooth muscle cells are cultured in adjacent compartments. Once the separating walls are removed, cells growing in different compartments will interact in a defined area.

### EXAMPLE 3

Cell Migration Studies

Cell mobility is one of the important features of cancer cells, which indicates the metastatic potential of a given cancer cell. Relative migration study refers to those studies comparing and understanding whether a certain type of cell behaves differently while migrating towards different types of cells (see FIG. 9). Currently, the standard experiment of measuring cell mobility is to scratch a line of certain width on the inner surface of a culture container where cells are growing, and measure the cell migration time over the scratched area. This method has a major disadvantage, which is only one type of cell is present in the process of migration. In other words, it is impossible for the current methods to study relative migrations of different types of cells, which is crucial for cancer invasion through cell barriers of different tissues.

The present co-culture system provides an ideal tool to study relative migrations of different type of cells under the same conditions. The migration speed can be monitored and measured in defined interacting areas. The design of the bottom edges of the partition unit (FIG. 9) allows relative migration experiments to provide not only information of migration speed, but also mutual growth impacts of different non-contacting cells. The mutual growth impacts between non-contacting cells refer to the mutual growth rate of two adjacent but non-contacting cells. For example, two different type of cells can grow in adjacent compartments separated by removable partition unit that has straight edge and sawtooth edge on its bottom edge. The three possibilities of growth impacts between cells including induction, inhibition, no-effect will result in different patterns of growth frontier lines that can be easily observed (see FIG. 9).

What is claimed is:

1. A cell co-culture system comprising a cell culture container partitioned into two or more compartments by one or more removable partition units, wherein the bottom edge of said removable partition unit(s) is in contact with the cell culture surface of said cell culture container, wherein different types of cells can be cultured in each of said compartments, and cellular interaction or cell migration is to be observed in an area on said cell culture container covered by the bottom edge(s) of said removable partition unit(s) after removal of said partition unit(s).

2. The cell co-culture system of claim 1, wherein said cell culture container is selected from the group consisting of a culture dish, a multi-well culture plate, and a culture chamber slide.

3. The cell co-culture system of claim 1, wherein said cell culture container is made of a material selected from the group consisting of polystyrene, glass, and plastic with high optical compatibility of glass.

4. The cell co-culture system of claim 1, wherein said cell culture container is treated or coated with biomembranes or biomolecules for the culture of specific cell type.

5. The cell co-culture system of claim 1, wherein the diameter or the diagonal dimension of said cell culture container is from about 10 mm to about 300 mm.

6. The cell co-culture system of claim 1, wherein said removable partition unit is made of a material selected from the group consisting of polystyrene, glass, medical grade silicon, metal, and biomembrane.

7. The cell co-culture system of claim 1, wherein the thickness of said removable partition unit is from about 0.01 mm to about 10 mm, wherein the width of the area covered by the bottom surface of said removable partition unit on said cell culture container is from about 0.01 mm to about 10 mm.

8. The cell co-culture system of claim 1, wherein each of the two sides on the bottom edge of said removable partition unit is in a shape selected from the group consisting of straight line, sawtooth-shaped and wave-shaped.

9. The cell co-culture system of claim 1, wherein the bottom edge of said removable partition unit is attached and sealed onto the cell culture surface of said cell culture container by sealing glue or pressure seal.

10. The cell co-culture system of claim 9, wherein said sealing glue or pressure seal is biologically inert, removable and leaves no damage to the cell culture surface of said cell culture container.

11. The cell co-culture system of claim 9, wherein said sealing glue or pressure seal comprises medical grade silicone glue or rubber.

12. The cell co-culture system of claim 1, wherein said cell culture container is a cell culture dish, and the opposite ends of each of said removable partition units are attached respectively to the wall of said culture dish and to a central culturing cylinder located in the middle of said cell culture dish, wherein the bottom edge of each of said removable partition units is attached to the cell culture surface of said culture dish by a sealing material that is connected to an overhang located on the exterior or interior side wall of said culture dish.

13. The cell co-culture system of claim 12, wherein the thickness of said removable partition unit is from about 0.01 mm to about 10 mm, and the width of the area covered by the bottom edge of said removable partition unit on said cell culture dish is from about 0.01 mm to about 10 mm.

14. The cell co-culture system of claim 12, wherein said sealing material is biologically inert, removable and leaves no damage to the cell culture surface of said cell culture dish.

15. The cell co-culture system of claim 14, wherein said sealing material comprises medical grade silicone glue or rubber.

16. The cell co-culture system of claim 1, wherein said removable partition unit is cylindrical in shape and encloses a circular area on said cell culture container.

17. The cell co-culture system of claim 16, wherein said circular area is further divided into two or more compartments by removable partition unit(s).

18. The cell co-culture system of claim 16, wherein said removable partition unit is attached to the cell culture surface of said cell culture container by direct and tight contact between optically polished surfaces of said partition unit and said cell culture container, said removable partition unit further comprises holder(s) that secure the position of said partition unit on said cell culture container.

19. The cell co-culture system of claim 16, wherein the thickness of said removable partition unit is from about 0.01 mm to about 10 mm, and the width of the area covered by the bottom edge of said removable partition unit on said cell culture container is from about 0.01 mm to about 10 mm.

20. The cell co-culture system of claim 1, wherein said removable partition unit is rectangular in shape and encloses a square or rectangular area on said cell culture container.

21. The cell co-culture system of claim 20, wherein said removable partition unit is attached to the cell culture surface of said cell culture container by direct and tight contact between optically polished surfaces of said partition unit and said cell culture container, said removable partition unit further comprises holder(s) that secure the position of said partition unit on said cell culture container.

22. The cell co-culture system of claim 20, wherein the thickness of said removable partition unit is from about 0.01 mm to about 10 mm, and the width of the area covered by the bottom edge of said removable partition unit on said cell culture container is from about 0.01 mm to about 10 mm.

23. The cell co-culture system of claim 20, wherein said square or rectangular area is further divided into two or more compartments by removable partition unit(s).

24. The cell co-culture system of claim 1, further comprising a cell sampler useful for collecting selected cells from said culture container, said cell sampler has a cutting end and a sampling end on its opposite ends, wherein said cutting end is used to mark a target region on the cultured cell layer, and said sampling end comprises a removable cell sampling surface to collect said selected cells.

25. The cell co-culture system of claim 24, wherein said cell sampling surface is a polycarbonate membrane or poly-lysine treated microscopic slide cover slip.

26. The cell co-culture system of claim 24, wherein said sampling end has a diameter or diagonal dimension from about 5 mm to about 50 mm.

27. The cell co-culture system of claim 24, wherein said cutting end and sampling end are in a shape selected from the group consisting of square, rectangle and circle.

28. A method of studying cell-cell interaction or interaction between a cell and a solid biologic material, said method comprises the steps of:
culturing different types of cells or solid biological material in the different compartments of the cell culture container of claim 1, wherein cells grow on the solid surface of said cell culture container partitioned by removable partition unit(s);
removing said removable partition unit(s) from said cell culture container; and
examining cell-cell interaction or interaction between a cell type and a solid biological material in the area covered previously by the bottom edge of said removable partition unit(s).

29. The method of claim 28, further comprises the steps of:
contacting a selected region on said cell culture container with the cutting end of a cell sampler, said cell sampler has a cutting end and a sampling end on its opposite ends;
collecting cells in said selected region with said sampling end of said cell sampler, wherein said sampling end comprise a removable cell sampling surface to which said cells in said selected region will adhere.

30. A method of studying cell-cell interaction or interaction between a cell and a solid biologic material, said method comprises the steps of:
culturing different types of cells or solid biological material in the different compartments of the cell culture container of claim 12, wherein cells grow on the solid surface of said cell culture container partitioned by removable partition unit(s);
removing said removable partition unit(s) from said cell culture container; and
examining cell-cell interaction or interaction between a cell type and a solid biological material in the area covered previously by the bottom edge of said removable partition unit(s).

31. The method of claim 30, further comprises the steps of:
removing the central culturing cylinder from said cell culture container; and
examining interaction among different types of cells in the area delimited previously by said central culturing cylinder.

32. The method of claim 30, further comprises the steps of:
contacting a selected region on said cell culture container with the cutting end of a cell sampler, said cell sampler has a cutting end and a sampling end on its opposite ends; and
collecting cells in said selected region with said sampling end of said cell sampler, wherein said sampling end comprise a removable cell sampling surface to which said cells in said selected region will adhere.

33. A method of studying relative migrations between different types of cells under the same conditions, said method comprises the steps of:
culturing different types of cells in the cell culture system of claim 1, wherein said cells are placed in different compartments of said cell culture container;
removing the removable partition unit(s) from said cell co-culture system; and
examining cell migrations towards the area covered previously by the bottom edge of said removable partition unit(s).

34. A method of studying mutual growth impacts of different types of live cells, said method comprises the steps of:
culturing different types of cells in the different compartments of the cell co-culture system of claim 1, wherein said compartments are partitioned by removable partition unit(s), wherein the bottom edge of said partition unit(s) is(are) sawtooth-shaped on one side and is(are) straight on the other side;
removing the removable partition unit(s) from said cell co-culture system; and
monitoring the interacting front lines of cells grown previously in adjacent compartments of said co-culture system.
